(19) United States
(12) Patent Application Publication
(10) Pub. No.: US 2009/0259123 A1
(43) Pub. Date: Oct. 15, 2009

(54) METHOD AND DEVICE FOR 3D ACQUISITION, 3D VISUALIZATION AND COMPUTER GUIDED SURGERY USING NUCLEAR PROBES

(75) Inventors: Nassir Navab, Munich (DE); Sibylle Ziegler, Munich (DE); Jörg Traub, Munich (DE); Thomas Wendler, Munich (DE)

Correspondence Address: PATTERSON & SHERIDAN, L.L.P., 3040 POST OAK BOULEVARD, SUITE 1500, HOUSTON, TX, 77056, US

(73) Assignee: SURGICEYE GMBH, Munich (DE)

(21) Appl. No.: 12/300,758

(22) PCT Filed: Feb. 27, 2007
(86) PCT No.: PCT/EP07/01678

§ 371 Date: Jan. 11, 2009

(30) Foreign Application Priority Data
May 16, 2006 (EP) 06010052.6

(51) Int. Cl. A61B 6/00

(52) U.S. Cl. 600/424; 600/436

(57) ABSTRACT

A device for 3D acquisition, 3D visualization and computer guided surgery with a tracking system, which can be for example an external optical tracking system (4), a tracked nuclear probe (1) having a nuclear probe (11) and a tip of nuclear probe (14), which measures a radioactivity, is achieved by providing a time synchronized recording of the spatial position and orientation of the tip of nuclear probes (14) and its measured counts of radioactivity. Thus a 3D radioactive surface distribution (8) is generated and may be visualized spatially registered with the viewing geometry of any in a superimposed image (81). Moreover using the said device, a method for 3D acquisition, 3D visualization and computer guided surgery is introduced for invasive or minimally invasive interventions. A further introduced method is the spatial localization of any surgical instrument in the same coordinate system as the said tracked nuclear probe (1) and the guidance of it to the said 3D radioactive surface distribution (8) during the surgical procedure. This is achieved by visualizing a simulated radioactivity measurement or feeding it backs in an acoustic signal. The said simulated radioactivity measurement is calculated based on the 3D radioactive surface distribution (8) and the position and orientation of the tip of the surgical instrument. The invention compromises also an integrated system that implements all or some of the said methods. Finally the invention includes a tracked combination of nuclear probe with camera (9).

The present invention relates to a method and to an device for 3D acquisition, 3D visualization and computer guided surgery using Nuclear Probes with a tracking system and a to localize malign tumorous cells of a human body or animal and a surgical instrument to remove the said diseased cells in both invasive and minimally invasive surgery.

The prior art related to the present invention are disclosed for example in U.S. Pat. No. 6,602,488, U.S. Pat. No. 6,456,869, U.S. Pat. No. 6,317,622 or U.S. Pat. No. 6,167,296 and allow trailing of the hand held probes as common diagnostic devices especially during surgery, as well as tracking systems for determination of position and orientation of surgical instruments. and imaging devices. Furthermore surface reconstruction is known as part of the state of the art.

The idea of tracking nuclear probes was mentioned in the past by several groups for example as disclosed in U.S. Pat. No. 6,510,336 and U.S. Pat. No. 6,021,341. However, these patents did not provide neither theory nor implementation nor application.

As further disclosed in U.S. Pat. No. 6,643,538 nuclear probes can be integrated per construction with a camera. However, this patent does not consider any kind of spatial localization.

Intraoperative probes have been used in the localization of tumors for 60 years. Lately beta emitting labeling has improved drastically their detection accuracy enabling a minimal, but complete resection of malignant cells and thus avoiding recurrence.

The output of nuclear probes is just a one dimensional signal usually not constant in time. The main advantages of such devices lie in the portability, simplicity, and the possibility of miniaturizing them for the investigation of cavities for instance mounted on endoscopes.

Moreover since each measurement is not restricted to be at a certain position with respect to the previous one, probes allow the scan of arbitrary surfaces with a spatial accuracy only limited by the size of the sensor.

Nuclear probes such as gamma- and beta-probes are capable of measuring radioactive decay of nuclides in tracers that are injected to the patient before the intervention.

The combination of nuclear probes with a camera in one device allows them further not only the determination of the radioactivity emitted by a certain region, but also the simultaneous visualization of the anatomy. This even allows the use of these nuclear probes to detect lesions through smaller incisions where the point of emission is only partially visible or completely occluded to the surgeon.

The disadvantage of these nuclear probes is that they are just point measurements. This makes the appreciation of the physical value on a surface difficult if it changes considerably with position. One additional problem in this matter is the fluctuation of the measurement results which is based on the statistical nature of the decay process which makes the interpretation of the measurement data even more difficult and unreliable. A further disadvantage is the need for many observations in order to get an idea of a valid measurement map in big areas, which is not the best solution for detection of hot spots in scans of big body sections.

Moreover, the sequential process of measuring and then performing the surgical action limits the accuracy to the surgeon's ability of navigating the instrument back to the detected locations.

Finally, in the specific case of the combination of nuclear probes with a camera one has a further disadvantage that the correlation of the measurement of the radioactivity to the anatomy seen in the video image is not given hence it has to be done in the mind of the surgeon.

The advantageous extending of the use of probes for combining position and orientation tracking with surface reconstruction, measurement and advanced visualization is not mentioned in the past.

The object of the present invention is to improve a computer guided surgery using Nuclear Probes of the above type such that the said disadvantages of the known arrangements are avoided and that in particular the disadvantage of nuclear probes performing point measurements, the difficulty of the interpretation of the measurement data due to its fluctuation, the further disadvantage namely the need for many observations in order to get an idea of a valid measurement map in big areas and finally the disadvantage of the sequential process of measuring and then performing the surgical action are compensated and thus a more reliable operation is guaranteed.

This invention also compensates the particular disadvantage of the combination of nuclear probes with a camera related to the lack of the correlation of the measurement of the radioactivity to the anatomy by including tracking systems and a proper visualization.

The advantageous extending of the use of probes for combining position and orientation tracking with surface reconstruction, measurement and advanced visualization is also to be implemented.

The invention achieves this object for a computer guided surgery using Nuclear Probes of the above type and tracking systems and in particular by first generating an activity surface in three dimensions by synchronized recording of the radioactivity and the position and orientation of a nuclear probe and thus making possible the interpretation of the measurements as a composite result; second visualizing this composite result which allows the operator to get a smooth impression of the activity distribution and thus to compensate the fluctuation; third calculating a mesh by using the measured positions and the a priori knowledge that they lie on a surface and further generating a proper interpolation of the results based on the said mesh; and finally presenting the generated surface activity distribution to the surgeon when applying the therapeutic actions and thus allowing the bridging of the time gap between measurement and action.

In a preferred embodiment of the invention the generated surface activity distribution is presented using an augmented reality system. In it, firstly, a visual, colour encoded surface model is superimposed on the real image of an external camera or a laparoscope; secondly, based on the recorded activity and the position and orientation of any tracked therapeutic device in the same coordinate system as the one in which the nuclear probe is tracked, a radioactivity measurement is simulated turning the surgical instrument into a virtual probe, and allowing the guidance of the surgeon to the areas of interest during surgical procedures.

The new technology can be used in both invasive and minimally invasive surgery and provides an end-to-end solution for minimal resection of malign tumorous cells reducing the remaining residual and therefore the risks of reoccurrence. It also results in more precise detection and treatment of afflicted lymph nodes.

A further improvement is the generating the same auditory feedback one gets during the nuclear probe examination, while approaching the same tissue with surgical instruments during surgical procedures.

In addition this invention solves the additional disadvantage of the combination of nuclear probes with a camera related to the lack of the correlation of the measurement of the radioactivity to the anatomy by the synchronized recording of the radioactivity, the camera image and the position and orientation of a nuclear probe. Based on the said synchronized recording, an activity surface in three dimensions is generated and it is projected onto the real image of the camera as a colour-encoded surface. Thus the correlation of the measurement of the radioactivity to the anatomy is given in a proper visualization.

The invention will now be elucidated by reference to the embodiment partially illustrated schematically in the drawings.

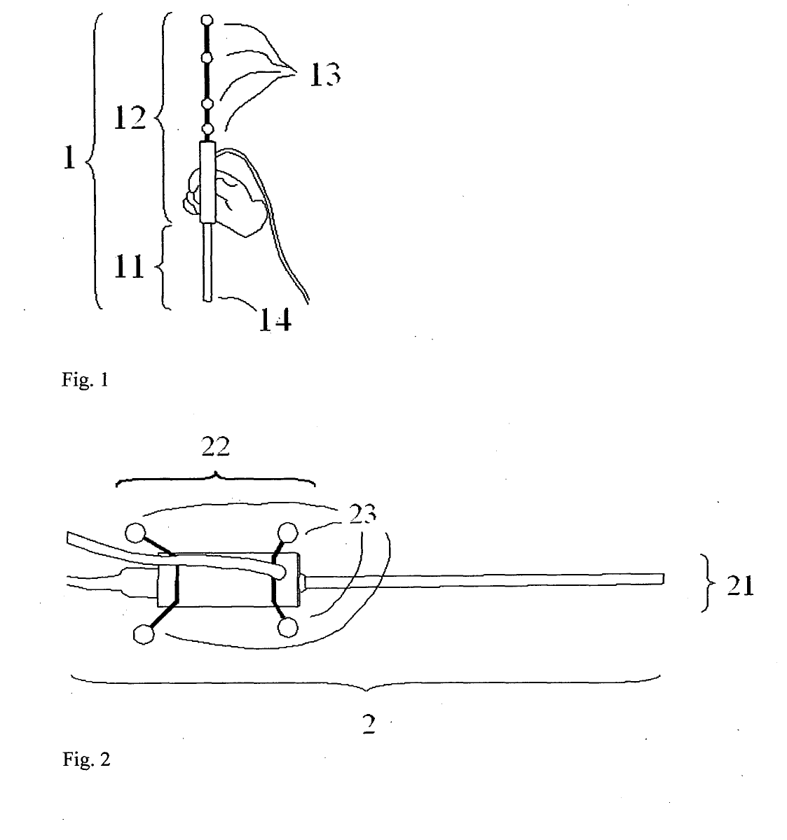

FIG. 1: a schematic view of a tracked nuclear probe

FIG. 2: a schematic view of a tracked laparoscope

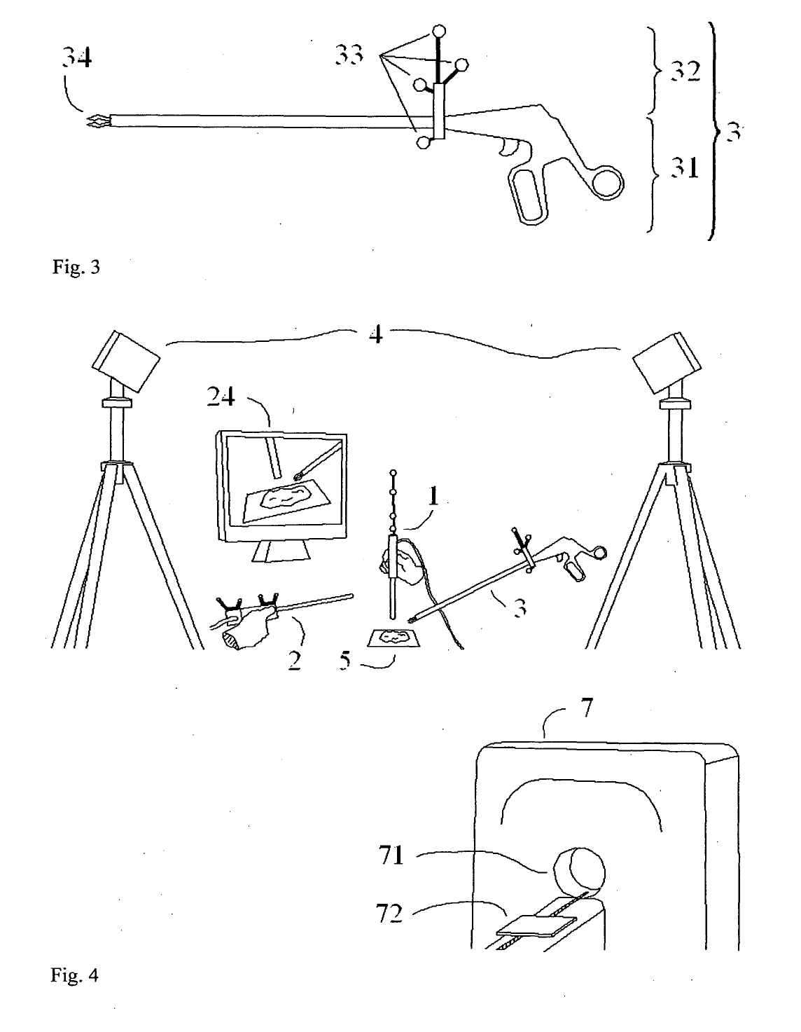

FIG. 3: a schematic view of a tracked surgical instrument

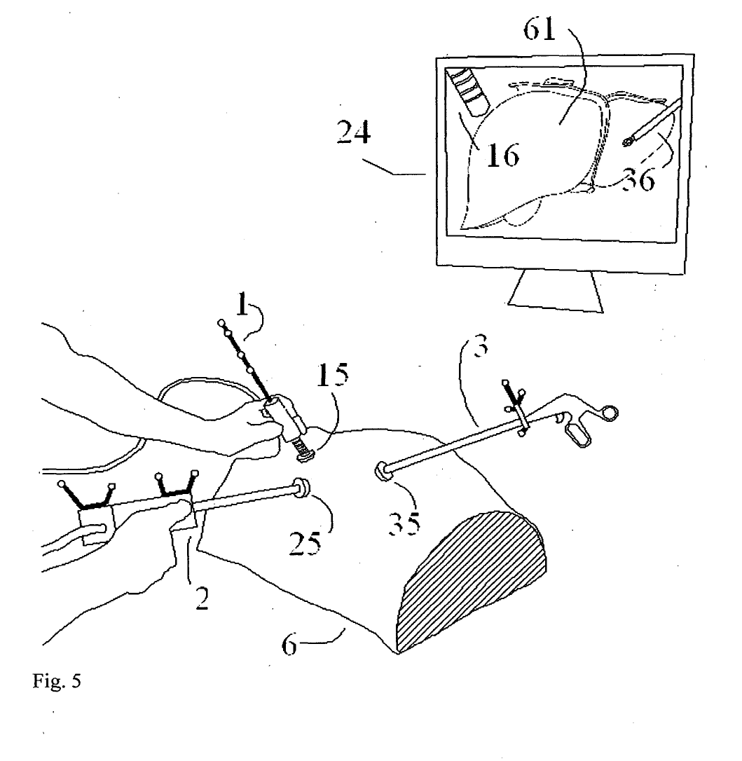

FIG. 4: a schematic view of an exemplary hardware set up for an experimental laparoscopic scan

FIG. 5: a schematic view of a laparoscopic scan on a human body with a laparoscope, a nuclear probe and a monitor

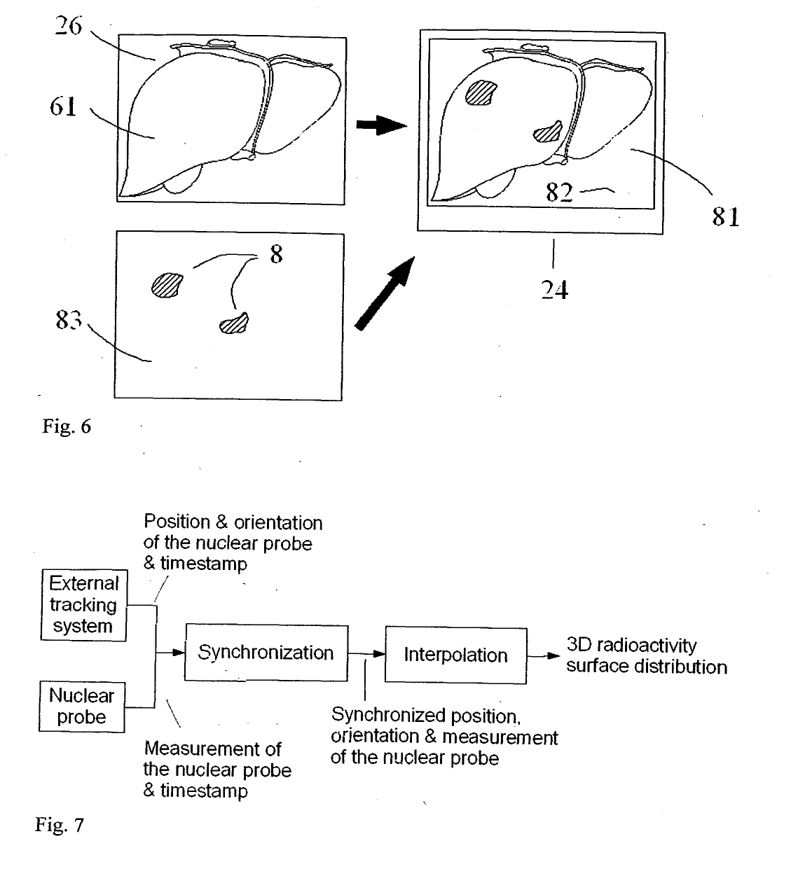

FIG. 6: schematic views of a body tissue, radioactive areas and a semi-transparent superposition of the radioactive areas to the body tissue

FIG. 7: a block diagram of a synchronized recording of the position and orientation measurement device

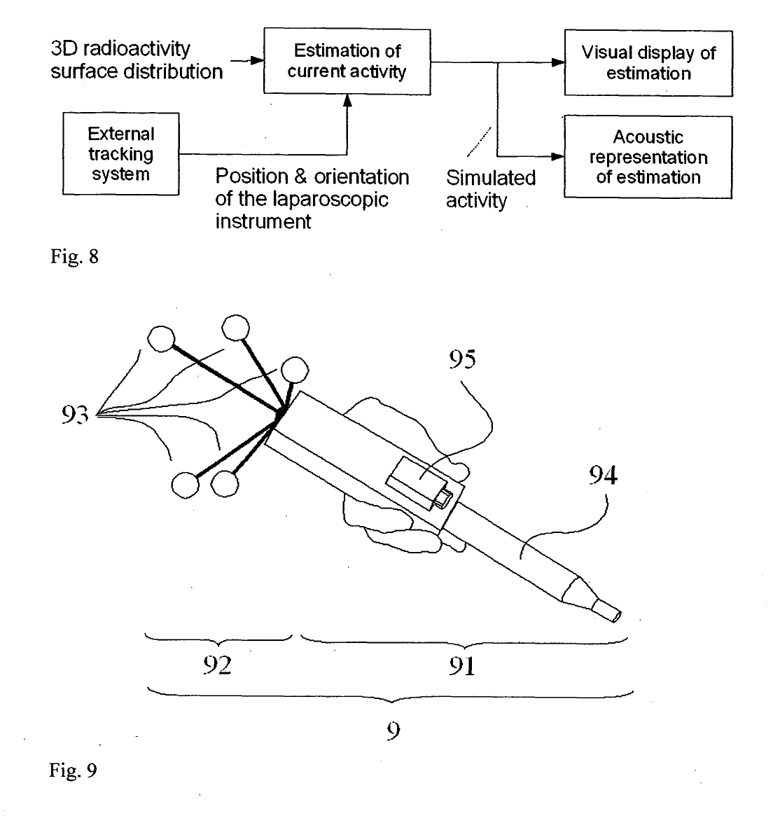

FIG. 8: presentation of a recorded activity by means of a tracked instrument

FIG. 9: schematic view of tracked combination of nuclear probe with camera

FIG. 1 shows a schematic of a tracked nuclear probe 1 which has essentially two parts, the first part is a nuclear probe 11 with a tip of nuclear probe 14 and the second part is an optical tracking target of nuclear probe 12 with an optical markers of nuclear probe 13. Based on the relative positions of the markers of nuclear probe 13, the position of the tip of the nuclear probe 14 and the probe axis can be estimated with a high precision in real time. The rotation around the axis of the nuclear probe 1 is not tracked, but it is also of no interest for the applications, since the measurement value is independent of this rotation. The readings of the nuclear probe 1 are sent to a proper data acquisition system for example a proper computer (not shown) using a data transfer means (not shown).

FIG. 2 shows a schematic view of a tracked instrument which is a tracked laparoscope 2 which has essentially two parts, the first part is a laparoscope 21 and the second part is an optical tracking target of laparoscope 22 which has optical markers of laparoscope 23. Based on the relative positions of the optical markers of laparoscope 23 the position and the orientation of laparoscope 21 can be estimated with a high precision in real time. Both the position and the orientation of laparoscope 21 are necessary to determine the correct view port of a laparoscopic video image 26, which is shown in FIG. 4.

FIG. 3 shows a schematic view of a tracked laparoscopic instrument 3 which is a tracked surgical instrument and has essentially two parts, the fist part is a laparoscopic instrument 31 with a tip of laparoscopic instrument 34 and the second part is an optical tracking target of laparoscopic instrument 32 with optical markers of laparoscopic instrument 33. Based on the relative positions of the markers of laparoscopic instrument 33, the position and orientation of the tip of the laparoscopic instrument 34 and axis of the laparoscopic instrument 31 can be estimated with a high precision in real time. In order to simulate the readings of the tracked nuclear probe 1 on the tip of laparoscopic instrument 34, both the position and the orientation of laparoscopic instrument 31 are necessary.

FIG. 4 shows an embodiment of a Device for 3D acquisition, 3D visualization and computer guided surgery using Nuclear Probes in an experimental case for a laparoscopic scan. A tracked nuclear probe 1 is used to acquire the 3D radioactive surface distribution 8 (shown in FIG. 6) of a phantom 5. An tracking system which can be for example an external optical tracking system 4 or an electro magnetic device (not shown) is used to track the tracked nuclear probe 1 as well as the tracked laparoscope 2 and the tracked laparoscopic instrument 3. The tracked laparoscope 2 acquires simultaneously a laparoscopic video image 26 (shown in FIG. 6). In order to visualized the acquired information a screen for image of laparoscope 24 is used. The said laparoscopic video image 26 is augmented with additional data like the 3D radioactive surface distribution 8. In case of the use of many cameras the video images of each of them can be augmented separately. A special case for that is for example a Head-Mounted Display where one image for each eye is augmented and thus a 3D impression is achieved. The tracked laparoscopic instrument 3 can also be tracked and the readings of the tracked nuclear probe 1 can be simulated on the tip of laparoscopic instrument 34. The simulated activity can be either displayed in the screen for image of laparoscope 24 or be fed back in form of acoustic signals that encode the 3D radioactive surface distribution 8. The encoding can be such that for a high radioactivity a high beep frequency and for a low radioactivity a low beep frequency is generated. A PET scanner 7 (Positron Emission Tomography) is used to validate the 3D radioactive surface distribution 8 in the phantom 5. Therefore the phantom 5 is positioned on a Phantom holder of PET scanner 72 and is to be moved into a gantry of PET scanner 71 to perform the imaging.

FIG. 5 shows a schematic view of a scan on a human body 6 with a tracked laparoscope 2, a tracked nuclear probe 1 and a screen for image of laparoscope 24 for scanning a body tissue 61. The tracked nuclear probe 1 is introduced through a proper trocar for tracked nuclear probe 15 into a human body 6 of a patient. The image of the inside is acquired using a tracked laparoscope 2 introduced by trocar for tracked laparoscope 25. Moreover the tracked laparoscopic instrument 3 can be introduced through a trocar for tracked laparoscopic instrument 35. A data acquisition system, for example a proper computer (not shown), integrates the laparoscopic video image 26 (shown in FIG. 6), the position and the orientation of laparoscope 21 and the position and orientation of the tip of the laparoscopic instrument 34 and axis of the laparoscopic instrument 31, where they are also synchronized with the readings of the tracked nuclear probe 1 and with the position and orientation of the tip of the nuclear probe 14. An image of the tip of nuclear probe on screen 16 as well as the body tissue 61 and an image of the tip of laparoscopic instrument 36 can be seen in the screen for image of laparoscope 24.

FIG. 6 shows schematic views of a body tissue 61, the laparoscopic video image 26, the 3D radioactive surface distribution 8, a projection of the said generated 3D radioactive surface distribution as a color encoded surface 83 and a semi-transparent superposition of 3D radioactive surface distribution 8 onto the body tissue 61 indicated as superimposed image 81. Additional information for example simulated radioactivity measurement 82 can be also shown on the screen for image of laparoscope 24. The simulated radioactivity measurement is calculated based on the 3D radioactive surface distribution 8 and the position and orientation of the tip of laparoscopic instrument 34. The simulated radioactivity measurement is to be equivalent to the one displayed by a nuclear probe 11 when detecting radioactivity.

FIG. 7 shows a block diagram of a synchronized recording of the position and orientation measurement device and the activity measures resulting in the generation of the three dimensional surface map that is presented to the surgeon. The external tracking system 4 feeds the data acquisition system with the position and orientation of the tip of the nuclear probe 14 as well as the timestamp of that measurement. Simultaneously the measurement of the nuclear probe 11 is also acquired with the timestamp of that measurement. Both the position and the orientation of the tip of the nuclear probe 14 and the measurement of the nuclear probe 11 are stored and synchronized. The synchronized position, orientation and measurement of the nuclear probe 11 are further interpolated to generate a 3D radioactive surface distribution 8.

FIG. 8 shows a presentation of a recorded activity by means of a tracked laparoscopic instrument 3. This could result either in a visual presentation such as an augmented reality view for example as a superimposed image 81 or in an acoustic representation simulating the real sound or count visualization of the nuclear probe 11. The optical external tracking system 4 feeds the data acquisition system with the position and orientation of the tip of laparoscopic instrument 34. The 3D radioactive surface distribution 8 calculated as described in FIG. 7 can then be integrated to generate a simulated activity on the tip of laparoscopic instrument 34. Based on the simulated activity the proper visualization or acoustic feedback can be calculated.

FIG. 9 shows a preferred embodiment of a tracked combination of a nuclear probe with a camera 9 which has essentially two parts. The fist part is a combination of a nuclear probe with a camera 91 with a nuclear probe of combination of a nuclear probe with a camera 94 and an optical camera system, which can be for example only one camera of combination of a nuclear probe with a camera 95 or a light guide that transmits the optical image to a camera connected to the device (not shown). The second part is an optical tracking target of combination of a nuclear probe with a camera 92 with optical markers of combination of a nuclear probe with a camera 93. Based on the relative positions of the markers of combination of a nuclear probe with a camera 93, the position and orientation of the nuclear probe of combination of a nuclear probe with a camera 94 and the position and orientation of the optical camera system can be estimated with a high precision in real time. Based on the synchronized radioactivity measurements of the nuclear probe of combination of a nuclear probe with a camera 94 and the position and orientation of the nuclear probe of combination of a nuclear probe with a camera 94, a 3D radioactive surface distribution 8 can be also generated. With the 3D radioactive surface distribution 8 a projection of the generated 3D radioactive surface distribution as a colour encoded surface 83 can be generated using the viewing geometry of the optical camera system and shown also as a superimposed image 81.

### Reference List of Drawings

1 tracked nuclear probe (modified beta-probe)

11 nuclear probe

12 optical tracking target of nuclear probe

13 optical markers of nuclear probe

14 tip of nuclear probe

15 trocar for tracked nuclear probe

16 image of the tip of nuclear probe on screen

2 tracked laparoscope

21 laparoscope

22 optical tracking target of laparoscope

23 optical markers of laparoscope

24 screen for image of laparoscope

25 trocar for tracked laparoscope

26 laparoscopic video image

3 tracked laparoscopic instrument

31 laparoscopic instrument

32 optical tracking target of laparoscopic instrument

33 optical markers of laparoscopic instrument

34 tip of laparoscopic instrument

35 trocar for tracked laparoscopic instrument

36 image of the tip of laparoscopic instrument

4 external optical tracking system

5 phantom

6 Human body

61 body tissue

7 PET scanner

71 Gantry of PET scanner

72 Phantom holder of PET scanner

8 3D radioactive surface distribution

81 superimposed image

82 additional information for example simulated radioactivity measurement

83 projection of the generated 3D radioactive surface distribution as a color encoded surface

9 tracked combination of a nuclear probe with a camera

91 combination of a nuclear probe with a camera

92 optical tracking target of nuclear probe of combination of a nuclear probe with a camera

93 optical tracking markers of nuclear probe of combination of a nuclear probe with a camera

94 nuclear probe of combination of a nuclear probe with a camera

95 camera of combination of a nuclear probe with a camera

1. A device for 3D acquisition, 3D visualization and computer guided surgery with an tracking system 4, a tracked nuclear probe 1 having a nuclear probe 11 and a tip of nuclear probe 14, which measures a radioactivity,
characterized in that a time synchronized recording of the spatial position and orientation of the tip of nuclear probe 14 and its measured counts of radioactivity is provided.

2. The device for 3D acquisition, 3D visualization and computer guided surgery according to claim 1,
characterized in that a 3D radioactive surface distribution 8, which can be a colour encoded surface 83, is generated using the said time synchronized spatial position and orientation of the tip of nuclear probe 14 with the said associated radioactivity measurements.

3. The device for 3D acquisition, 3D visualization and computer guided surgery according to claim 2,
characterized in that a projection of the generated 3D radioactive surface distribution as a colour encoded surface 83 is visualized spatially registered with the viewing geometry of any camera or set of cameras for example a laparoscope 21 or Head-Mounted Display in a superimposed image 81 or a set of superimposed images 81.

4. A method for 3D acquisition, 3D visualization and computer guided surgery using the device according to any of the preceding claims,
characterized in that the 3D radioactive surface distribution 8 or the superimposed image 81 or the projection of the generated 3D radioactive surface distribution as a colour encoded surface 83 or any combination of them are visualized and used for invasive or minimally invasive interventions.

5. A method for 3D acquisition, 3D visualization and computer guided surgery using the device according to any of the preceding claims,
characterized in that intraoperative measured counts of radioactivity of the nuclear probe 11 are directly augmented onto a camera video image for example a laparoscopic video image 26.

6. A method for 3D acquisition, 3D visualization and computer guided surgery using the device according to any of the preceding claims,
characterized in that any surgical instrument, for example a laparoscopic instrument 31, is spatially localized in the same coordinate system as the said tracked nuclear probe 1 and the said surgical instrument is guided to the said 3D radioactive surface distribution 8 during the surgical procedure.

7. A method for 3D acquisition, 3D visualization and computer guided surgery using the device according to any of the preceding claims,
characterized in that a simulated radioactivity measurement is calculated based on the 3D radioactive surface distribution 8 and the position and orientation of the tip of a surgical instrument for example the tip of laparoscopic instrument 34 and displayed, while navigating any tracked surgical instruments, for example a tracked laparoscopic instrument 3, over the previously generated 3D radioactive surface distribution 8.

8. A method for 3D acquisition, 3D visualization and computer guided surgery using the device according to any of the preceding claims,
characterized in that a simulated radioactivity measurement is calculated based on the 3D radioactive surface distribution 8 and the position and orientation of the tip of a surgical instrument for example the tip of laparoscopic instrument 34 and be fed back in form of acoustic signals, while navigating any tracked surgical instruments, for example a tracked laparoscopic instrument 3, over the previously generated 3D radioactive surface distribution 8.

9. The device for 3D acquisition, 3D visualization and computer guided surgery according to any of preceding claims,
characterized in that a system is provided which integrates
a module for the visualization of the 3D radioactive surface distribution 8 or the superimposed image 81 or the projection of the generated 3D radioactive surface distribution as a colour encoded surface 83 or any combination of them for invasive or minimally invasive interventions,
a module for the direct augmentation of intraoperative measured counts of radioactivity of the nuclear probe 11 onto a camera video image for example a laparoscopic video image 26,
a module for the spatial localization of any surgical instrument, for example a laparoscopic instrument 31, in the same coordinate system as the said tracked nuclear probe 1 and the guidance of the said surgical instrument to the said 3D radioactive surface distribution 8 during the surgical procedure,
a module for the calculation and display of a simulated radioactivity measurement is calculated based on the 3D radioactive surface distribution 8 and the position and orientation of the tip of a surgical instrument for example the tip of laparoscopic instrument 34, while navigating any tracked surgical instruments, for example a tracked laparoscopic instrument 3, over the previously generated 3D radioactive surface distribution 8 and
a module for the calculation and feedback in form of acoustic signals of a simulated radioactivity measurement is calculated based on the 3D radioactive surface distribution 8 and the position and orientation of the tip of a surgical instrument for example the tip of laparoscopic instrument 34, while navigating any tracked surgical instruments, for example a tracked laparoscopic instrument 3, over the previously generated 3D radioactive surface distribution 8,
or any combination of any of the modules in a software framework that provides a unified user interface for visualization of intraoperative measured counts of radioactivity.

10. The device for 3D acquisition, 3D visualization and computer guided surgery according to any of preceding claims,
characterized in that a tracked combination of nuclear probe with camera 9 having a combination of nuclear probe with camera 91, which has a nuclear probe of combination of a nuclear probe with a camera 94 and an optical camera system is provided.
